(19) United States
(12) Patent Application Publication
(10) Pub. No.: US 2017/0207015 A1
(43) Pub. Date: Jul. 20, 2017

(54) MAGNETIC CIRCUIT FOR PRODUCING A CONCENTRATED MAGNETIC FIELD

(71) Applicants: MONASH UNIVERSITY, Clayton, Victoria (AU); ALFRED HEALTH, Melbourne, Victoria (AU)

(72) Inventors: Malin PREMARATNE, Camberwell, Victoria (AU); Chintha HANDAPANGODA, Glen Waverley, Victoria (AU); Paul FITZGERALD, Hampton, Victoria (AU); Philip LEWIS, Melbourne, Victoria (AU); Richard THOMSON, Armadale, Victoria (AU)

(73) Assignees: MONASH UNIVERSITY, Victoria (AU); ALFRED HEALTH, Victoria (AU)

(21) Appl. No.: 15/315,515

(22) PCT Filed: Jun. 2, 2015
(86) PCT No.: PCT/AU2015/050300

§ 371 Date: Dec. 1, 2016

(30) Foreign Application Priority Data
Jun. 2, 2014 (AU) 2014902109

(51) Int. Cl. H01F 7/20; A61N 2/02; A61N 2/00; H01F 7/06; H01F 1/147

(52) U.S. Cl. CPC H01F 7/20; H01F 7/064; H01F 1/14708; A61N 2/006; A61N 2/02

(57) ABSTRACT

A magnetic circuit (400) comprises a magnetic path which includes at least one magnetic source (102) arranged to generate magnetic flux within the magnetic path. A magnetic flux-concentrating element 402 is magnetically coupled to the magnetic source, and arranged to concentrate magnetic-flux generated by the magnetic source within a volume adjacent to the magnetic flux-concentrating element (402). Some embodiments of the magnetic circuit (400) comprise at least first (102) and second (104) magnetic sources, which may advantageously be arranged to generate magnetic flux in a common direction within the magnetic path.

### FIELD

Field of the Invention

The present invention relates to magnetic circuits. More particularly, embodiments of the invention comprise magnetic circuits for concentrating or focusing magnetic fields within a localised volume. Applications of the invention include magnetic stimulation and magnetic lensing.

Background to the Invention

Time-varying magnetic fields are employed in a number of applications. For example, beams of moving charged particles, such as electrons, can be focused, controlled and deflected in a desired manner using spatially and time-varying magnetic fields. Such arrangements, also known as magnetic lenses, are employed in systems such as transformers, electric motors, cathode ray tubes, electron microscopes, and particle accelerators.

Another use of time-varying magnetic fields is Transcranial Magnetic Stimulation (TMS), which involves the application of magnetic fields to depolarise or hyper-polarise neurons within the brain. TMS is used in neuroscience and clinical applications in psychiatry and neurology. All current TMS systems use coils placed outside of the head to induce activity within the brain.

There are limitations to the way in which TMS is currently provided. Repeated stimulation sessions over a number of weeks are usually required to improve the symptoms. Patients are required to attend a clinical environment on a daily basis for up to six to eight weeks. Even then, a significant percentage of patients relapse in the following months, requiring repeated treatment. In these conventional systems a large (approximately 7 cm-wide) external coil is used to induce neural activity via magnetic stimulation. With these coils, an undesirably large area of the brain is stimulated.

Other forms of magnetic bio-stimulation include neural muscular stimulation, such as renal stimulation.

All of the aforementioned applications, along with many others such as electric motors, magnetic confinement systems, and so forth, can benefit from improved control of the region (i.e. volume of space) within which magnetic fields are produced. Generating more highly concentrated magnetic fields where desired, while minimising the generation of fields in unwanted regions, improves efficiency and reduces power consumption. Reducing power consumption, in turn, reduces heating. In the case of neural stimulation, providing a more focused magnetic field permits more selective stimulation, spatially and functionally.

International Patent Application Publication No. WO 2014/016073, in the name of Universitat Autonoma de Barcelona, and published on 30 Jan. 2014, discloses a device for concentrating or amplifying magnetic flux. However, the device is designed only to concentrate an existing magnetic field. It does not enable a field to be generated, or modulated, in addition to being focused.

In Bonmassar, G, et al, ‘Microscopic Magnetic Stimulation of Neural Tissue,’ published in Nature Communications, Volume 3, 2012, page 921, a miniaturised coil system is disclosed, in which conductive coils are implanted on the retinal surface in order to provide ongoing localised neural stimulation. The authors further propose the use of this type of device, implanted into brain tissue, for cortical neural stimulation. However, this approach would benefit from greater control and localisation of the magnetic field. In particular, it would be desirable to generate the required field strength at a target location using a reduced power input, in order to minimise heating of the implant.

An object of the present invention is therefore to provide an improved magnetic circuit for producing a concentrated magnetic field with enhanced localisation, and reduced input power requirements, when compared with the prior art discussed above.

### SUMMARY OF THE INVENTION

According to an aspect of the invention, there is provided a magnetic circuit comprising a magnetic path including:

at least one magnetic source arranged to generate magnetic flux within the magnetic path; and

at least one magnetic flux-concentrating element, magnetically coupled to the magnetic source, and arranged to concentrate magnetic-flux generated by the magnetic source within a volume adjacent to the magnetic flux-concentrating element.

Advantageously, embodiments of the invention are able to generate magnetic fields, and to concentrate (focus) those magnetic fields into a desired volume. This enhances an induced magnetic field in a target area, while minimising the spread to other areas. When used for neural stimulation, for example, desired magnetic field intensity may be achieved within the target volume, with reduced power consumption, heating, and undesired neural stimulation of surrounding areas.

The reduced size, improved focussing and reduced power consumption of embodiments of the invention, relative to prior art magnetic circuits, makes them well-suited to use as implantable bio-stimulation devices. Embodiments of the invention may also be advantageously used externally. For example, compared with prior art external TMS coils, which can be bulky, have high power consumption, and may generate considerable heat requiring liquid or other cooling designs, embodiments of the present invention may be smaller, exhibit higher energy efficiency, and produce less heat.

In embodiments of the invention, the at least one magnetic source comprises at least first and second magnetic sources. The first and second magnetic sources may comprise, for example, conductive coils through which an electric current is passed, in order to induce a magnetic field. The magnetic field so produced may be time-varying, for example by applying an alternating current input to the conductive coils. Preferably, the coils are formed around a core comprising a magnetic material, such as a ferromagnetic material.

In some embodiments of the invention the magnetic flux-concentrating element comprises first and second tapered portions, each formed from a magnetic material and having a wider end and a narrower end, wherein the wider end of the first tapered portion is magnetically coupled to the first magnetic source, the wider end of the second tapered portion is magnetically coupled to the second magnetic source, and the narrower ends of the first and second tapered portion converge within a volume in which magnetic flux is concentrated.

In some embodiments comprising first and second tapered portions, the concentrating element further includes at least one diamagnetic portion located adjacent to the first and second tapered portions.

In some embodiments, the first and second magnetic sources are arranged to generate magnetic flux in a common direction within the magnetic path. Advantageously, the inventors have found that, in at least some embodiments of the invention, greater intensity of the concentrated magnetic fields is obtained when the fields generated by the magnetic sources propagate in a common direction.

In embodiments of the invention, the first and second magnetic sources are magnetically coupled by a continuous length of magnetic material defining a portion of the magnetic path. More particularly, the continuous length of magnetic material may define a portion of the magnetic path comprising a substantially 180 degree change of direction of magnetic flux. In particular embodiments, the continuous length of magnetic material comprises a substantially ‘U’-shaped section.

According to some embodiments, the magnetic flux-concentrating element comprises a structure forming an arc having an inner surface and an outer surface wherein:

the first and second magnetic sources are magnetically coupled to the outer surface of the structure; and

the structure comprises an alternating arrangement of magnetic and diamagnetic materials forming layers extending between the outer surface and the inner surface.

In some embodiments, the arc is a substantially circular arc, such that the inner surface is defined by an inner radius, and the outer surface is defined by an outer radius. The arc may be an open arc, such that the structure is substantially ‘C’-shaped.

It should be noted that, within this specification, the term ‘diamagnetic’ encompasses materials that are at least partially diamagnetic, at least under certain temperature conditions. Diamagnetic materials include, in particular, superconducting materials, whether low temperature (1.5 to 5 kelvin) or high temperature (up to and beyond 110 kelvin) super conductors. Diamagnetic materials also include strongly diamagnetic elements, such as bismuth. Diamagnetic materials also include engineered material such as pyrolytic carbon.

As will be appreciated, new diamagnetic materials, including superconducting materials, are discovered and/or developed with continuing regularity, and constitute an active and ongoing area of research and development. The principles of operation of the invention do not depend upon specific details of the composition of the magnetic or diamagnetic materials employed, and it is envisaged that structures embodying the invention can be manufactured using presently available magnetic and diamagnetic materials, as well as new materials that will become available in the future.

In some embodiments the diamagnetic material comprises pyrolytic carbon. Advantageously, pyrolytic carbon is strongly diamagnetic, and is also biocompatible. Of materials currently known, it exhibits the greatest diamagnetism (by weight) of any room-temperature diamagnetic material. Calculations performed by the inventors indicate that the efficiency of a flux-concentrating element comprising pyrolytic carbon may be within 30% of the maximum efficiency achievable using a superconducting material. Pyrolytic carbon is thus presently the best candidate material for use in implantable devices, such as neural biostimulation devices, due to its biocompatibility and efficiency of operation at ambient body temperatures.

In another aspect, the invention provides a method of generating a magnetic field which comprises:

providing a magnetic circuit as previously described; and

applying driving electrical signals to the first and second magnetic sources in order to generate magnetic flux within the magnetic path, whereby a concentration of magnetic flux is generated within a volume adjacent to the magnetic flux-concentrating element.

Further features and advantages of embodiments of the invention will be apparent to persons skilled in the art from the following description of specific embodiments, which is provided by way of example to assist in understanding of the principals of the invention, but should not be taken to limit the scope of the invention as defined in any of the preceding statements, or in the claims appended hereto.

### BRIEF DESCRIPTION OF THE DRAWINGS

Embodiments of the invention will now be described with reference to the accompanying drawings, in which like reference numerals indicate like features, and wherein:

FIG. 1 shows a first magnetic circuit embodying the invention;

FIG. 2 shows a second magnetic circuit embodying the invention;

FIG. 3 shows a third magnetic circuit embodying the invention;

FIG. 4 shows a fourth magnetic circuit embodying the invention;

FIGS. 5(a) to (d) illustrates magnetic field intensity within a volume adjacent to the magnetic circuit of FIG. 4;

FIG. 6 is a graph illustrating magnetic flux density at various points adjacent to the magnetic circuit of FIG. 4, as compared with a comparable microcoil implant;

FIG. 7 is a graph illustrating magnetic energy within various small volumes adjacent to the magnetic circuit of FIG. 4, as compared with a comparable microcoil implant;

FIG. 8 is a graph illustrating maximum magnetic flux density adjacent to the magnetic circuit of FIG. 4, as compared with a comparable microcoil implant, as a function of excitation current;

FIG. 9 is a graph illustrating maximum magnetic energy density adjacent to the magnetic circuit of FIG. 4, as compared with a comparable microcoil implant, as a function of excitation current;

FIG. 10 is a graph illustrating maximum temperature as a function of time over which continuous current excitation is applied to the magnetic circuit of FIG. 4;

FIG. 11(a) is a graph illustrating maximum temperature as a function of applied current excitation to the magnetic circuit of FIG. 4, over a fixed duration;

FIG. 11(b) is a graph showing temperature as a function of time during application of trains of repetitive current pulses to the magnetic circuit of FIG. 4;

FIG. 12 is a graph illustrating magnetic field penetration depth as a function of applied current excitation, for the magnetic circuit of FIG. 4; and

FIGS. 13(a) to 13(f) illustrate a number of alternative embodiments of the invention.

### DETAILED DESCRIPTION OF EMBODIMENTS

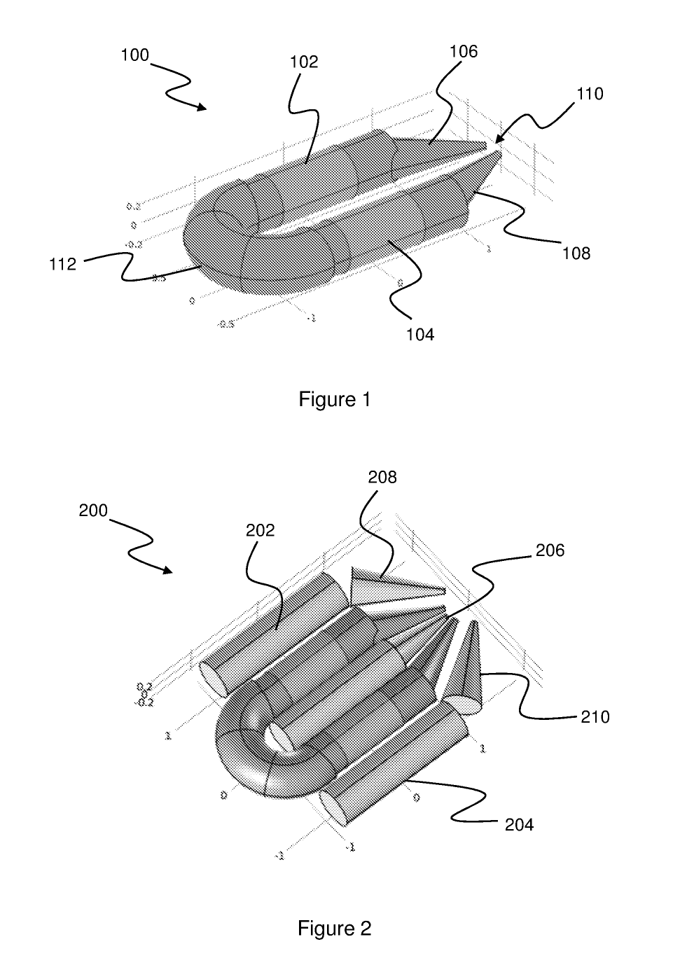

FIG. 1 illustrates a first magnetic circuit 100 embodying the present invention. The magnetic circuit 100 comprises first and second magnetic sources, 102, 104, each of which consists of a conductive coil wound around a magnetic core.

The magnetic circuit 100 further comprises magnetic flux-concentrating elements 106, 108, which are magnetically coupled to the first and second magnetic sources 102, 104. In the magnetic circuit 100, the magnetic flux-concentrating elements 106, 108 are fabricated from the same magnetic material as the cores of the magnetic sources 102, 104, and may be formed integrally, or joined after fabrication, in order to provide magnetic coupling.

Each of the magnetic flux-concentrating elements 106, 108 comprises a tapered portion which has a wider end coupled to the associated magnetic source and an opposing narrower end. As shown, the two tapered flux-concentrating elements 106, 108 converge within a small volume 110 in which magnetic flux is concentrated when the magnetic sources 102, 104 are activated.

Additionally, the first and second magnetic sources 102, 104 of the circuit 100 are coupled by a continuous portion of magnetic material 112.

Overall, the magnetic circuit 100 comprises a magnetic path including the first and second magnetic sources 102, 104, the connecting portion of magnetic material 112, the flux-concentrating elements 106, 108, and the air gap within the small concentrating volume 110.

As will be appreciated, activation of the magnetic sources 102, 104 is achieved by passing a current through the coils. By selecting the polarity of the current in each coil, the magnetic sources 102, 104 may be operated so as to generate magnetic flux in a common direction around the magnetic path formed by the magnetic circuit 100. Alternatively, the magnetic sources 102, 104 may be operated to direct magnetic flux in opposite directions around the magnetic circuit 100, for example such that magnetic flux is commonly directed towards the tapered ends of the magnetic flux-concentrating elements 106, 108. In practice, fields generated in opposing directions may repel one another, reducing the magnetic flux density within the target volume 110. Greater concentration of magnetic fields may therefore be achieved by operating the magnetic sources 102, 104 to generate magnetic flux in a common direction within the magnetic path formed by the circuit 100.

FIG. 2 shows a second magnetic circuit 200 embodying the invention. The magnetic circuit 200 comprises an embodiment of the first magnetic circuit 100, to which additional elements have been added. Whereas the first magnetic circuit 100 is formed from a core of magnetic material, joined to or integral with a length of coupling magnetic material 112 and magnetic flux-concentrating elements 106, 108, the additional elements of the magnetic circuit 200 are fabricated from a diamagnetic material. In particular, the diamagnetic material may be a superconducting material, or may be a strongly room-temperature diamagnetic material, such as pyrolytic carbon.

As shown in FIG. 2, diamagnetic elements are placed around the magnetic elements of the first magnetic circuit 100. Two diamagnetic elements 202, 204 are positioned on either side of the central magnetic circuit 100, a third diamagnetic element 206 is positioned in the centre of the magnetic circuit 100, i.e. between the magnetic sources 102, 104 along with the magnetic flux-concentrating elements 106, 108, and finally two further diamagnetic elements 208, 210 are positioned adjacent to the outer edges of the magnetic flux-concentrating elements 208, 210. As shown, the two diamagnetic elements 208, 210 are tapered in like manner to the magnetic flux-concentrating elements 106, 108.

The basic principle of operation of the second magnetic circuit 200 is that while magnetic elements strongly support the transmission of magnetic fields, diamagnetic elements effectively repel magnetic fields. Accordingly, the positioning of diamagnetic element within the circuit 200 is intended to further enhance concentration of the magnetic fields within the volume of space adjacent to the magnetic flux-concentrating elements 106, 108.

In particular, it is desirable that the magnetic materials comprising the circuit 100 have a high magnetic permeability, while the diamagnetic materials have a very low magnetic permeability. The magnetic material may be a moderately electrically conductive ferromagnetic material, and in particular may be a high magnetic permeability material such as permalloy (i.e. a ferromagnetic alloy formed from approximately 80 percent nickel, the remaining 20 percent being primarily iron, alloyed with smaller quantities of other elements such as carbon, manganese, silicon and molybdenum).

The diamagnetic material may be a superconducting material, having extremely high electrical connectivity and very low magnetic permeability. Depending upon the application, and practical operating temperatures, the superconducting material may be a high temperature superconductor (i.e. operating in a superconducting state at or above liquid nitrogen temperature of 77 kelvin), or may be a low temperature superconductor. Various ceramics are known to exhibit superconducting properties above the temperatures of liquid nitrogen, and materials exhibiting superconductivity at ever-increasing temperatures are regularly reported.

Alternatively, the diamagnetic may be a non-superconducting diamagnet, such as pyrolytic carbon. Advantageously, in the case of implantable devices, pyrolytic carbon exhibits diamagnetic properties at and above room temperature, and has good biocompatibility.

The coils of the magnetic sources may be formed from a number of turns of a conventional electrically conductive material, such as copper or silver.

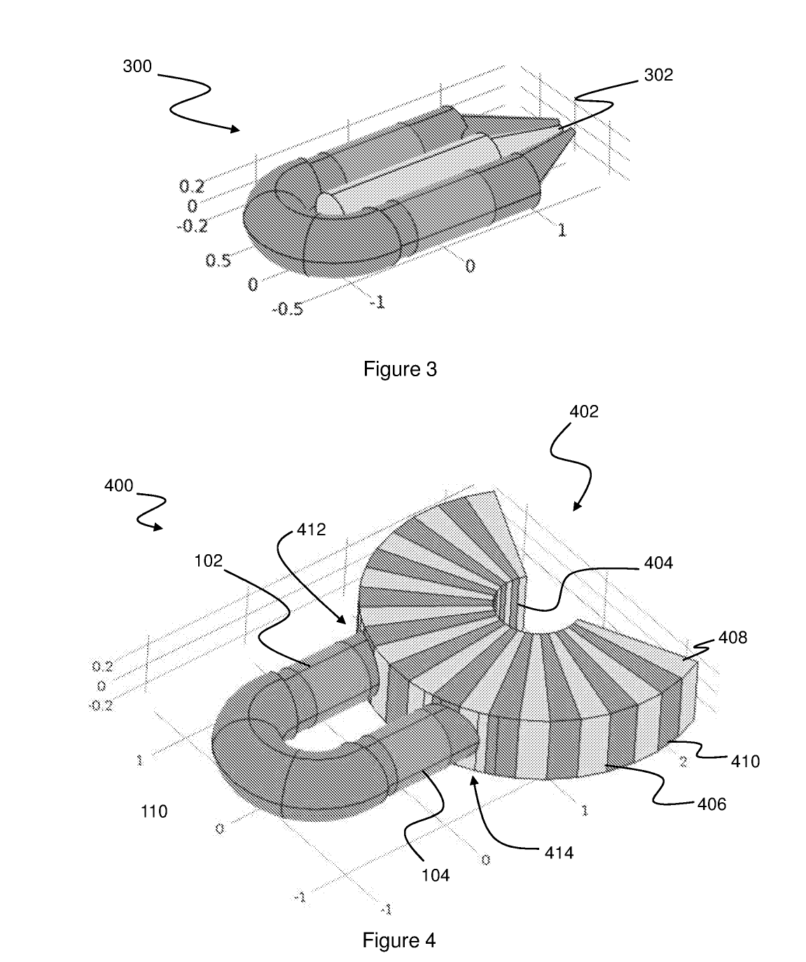

FIG. 3 shows a third magnetic circuit 300, which differs from the second magnetic circuit 200 in that the outer diamagnetic elements are omitted, and only a central diamagnetic element 302 is provided. Simulations of the second and third magnetic circuits 200, 300 conducted by the inventors have established that a very similar maximum magnetic flux is generated by the two circuits 200, 300, suggesting that only a minimal additional benefit is achieved through the inclusion of the external diamagnetic elements 202, 204, 206, 208.

FIG. 4 illustrates a fourth magnetic circuit 400 embodying the invention. As in the first to third magnetic circuits, the fourth magnetic circuit 400 comprises first and second magnetic sources 102, 104, each of which includes a magnetic core (such as permalloy), around which conductive coils are wound. The two magnetic sources 102, 104 are again also coupled via a U-shaped portion 110 of magnetic material, e.g. permalloy. However, the magnetic circuit 400 comprises a different form of magnetic flux-concentrating element 402 from the first and second tapered portions employed in the first to third circuits.

The magnetic flux-concentrating element 402 in the circuit 400 comprises a structure generally forming an arc having an inner surface 404 and an outer surface 406. In the particular circuit 400 the arc-shaped element 402 has the form of a segment of a cylindrical shell, wherein the inner surface 404 forms the interior surface of the partial cylinder, and the outer surface 406 forms the exterior surface of the partial cylinder. The arc is substantially circular, such that the inner surface is defined by an inner radius, and the outer surface is defined by an outer radius. Since the flux concentrating element 402 has the form of a segment of a cylinder, in plan view it forms an open arc which is substantially ‘C’-shaped in appearance.

Other related forms may be employed for the magnetic flux-concentrating element 402. For example, the element 402 may have the form of other solids of rotation. Alternatively, rather than having a circular cross-section the magnetic flux-concentrating element 402 may have an ovoid cross-section.

The structure of the magnetic flux-concentrating element 402 comprises an alternating arrangement of diamagnetic (e.g. 408) and magnetic (e.g. 410) materials, comprising a series of layers extending (radially) between the outer surface 406 and the inner surface 404 of the magnetic flux-concentrating element 402.

The first and second magnetic sources 102, 104 are magnetically coupled to the outer surface 406 of the magnetic flux-concentrating element 402. As illustrated in FIG. 4, according to the embodiment 400 the core of the magnetic sources 102, 104 extends towards, and is joined to, the outer surface 406 of the magnetic flux-concentrating element 402 at corresponding locations 412, 414.

The fourth magnetic circuit 400 embodying the invention employs a magnetic flux-concentrating element 402 having similar principals of operation to the magnetic flux concentrator disclosed in International Publication No. WO 2014/016073.

The inventors have conducted a number of computer-based calculations/simulations of performance of magnetic circuits embodying the design 400 shown in FIG. 4. In these calculations, the magnetic material is assumed to be permalloy with a relative permeability of 1000, while the diamagnetic material is assumed to be superconducting material with a relative permeability of 0.0001. The electrical conductivity of the superconducting material is assumed to be 1×10¹⁰ S/m, while the conductors forming the coils are assumed to be made of copper, with a conductivity of 6×10⁷S/m.

In order to clearly illustrate the principles and performance of the circuit 400, results based on the use of a superconducting diamagnetic material are discussed below. However, further calculations/simulations conducted by the inventors have established that the use of pyrolytic carbon achieves similar results, with a loss in efficiency of only around 30%. Thus the results may be considered representative of various embodiments of the invention employing a range of different diamagnetic materials.

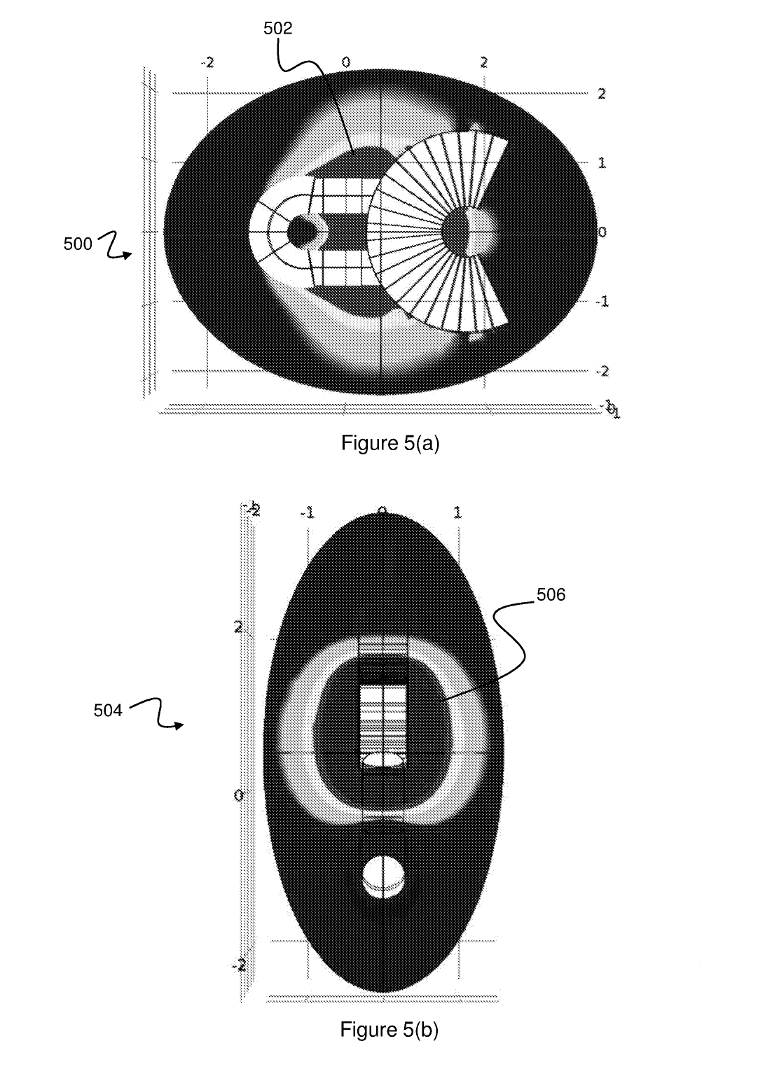

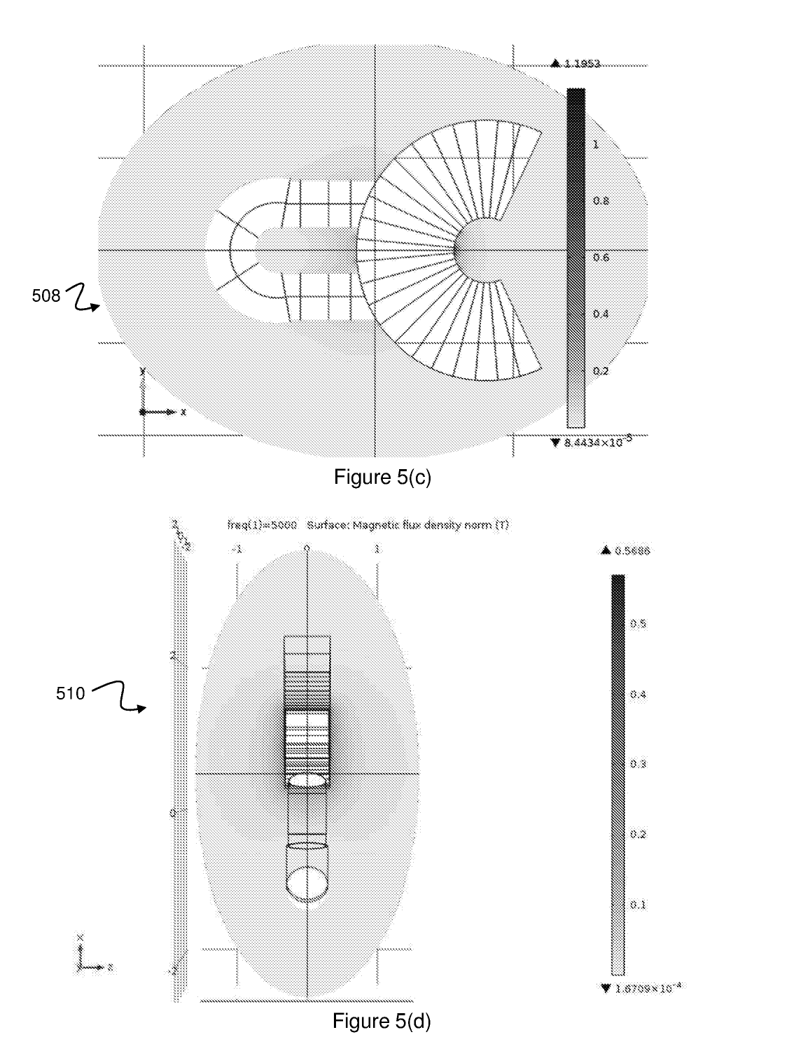

FIG. 5 illustrates magnetic field intensity within a volume adjacent to the magnetic circuit, having the general design shown in FIG. 4, with 21 turns of wire in each coil, and applying a coil current of one ampere. In each case, a central shaded zone adjacent to the magnetic circuit illustrates a volume within which the magnetic field exceeds a threshold level of 0.02T, which corresponds with a sufficient intensity to induce a neural response in a magnetic stimulation application.

FIG. 5(a) illustrates firstly a top view 500 of the magnetic circuit showing a region 502 in the plane of the circuit where the magnetic field strength exceeds the threshold level. A side/cross-sectional view 504 in FIG. 5(b) shows the corresponding region 506 extending above and below the magnetic circuit. FIGS. 5(c) and 5(d) show the corresponding original results 508, 510, without the threshold colouring, and show more clearly the focusing and spread of the magnetic field around the circuit.

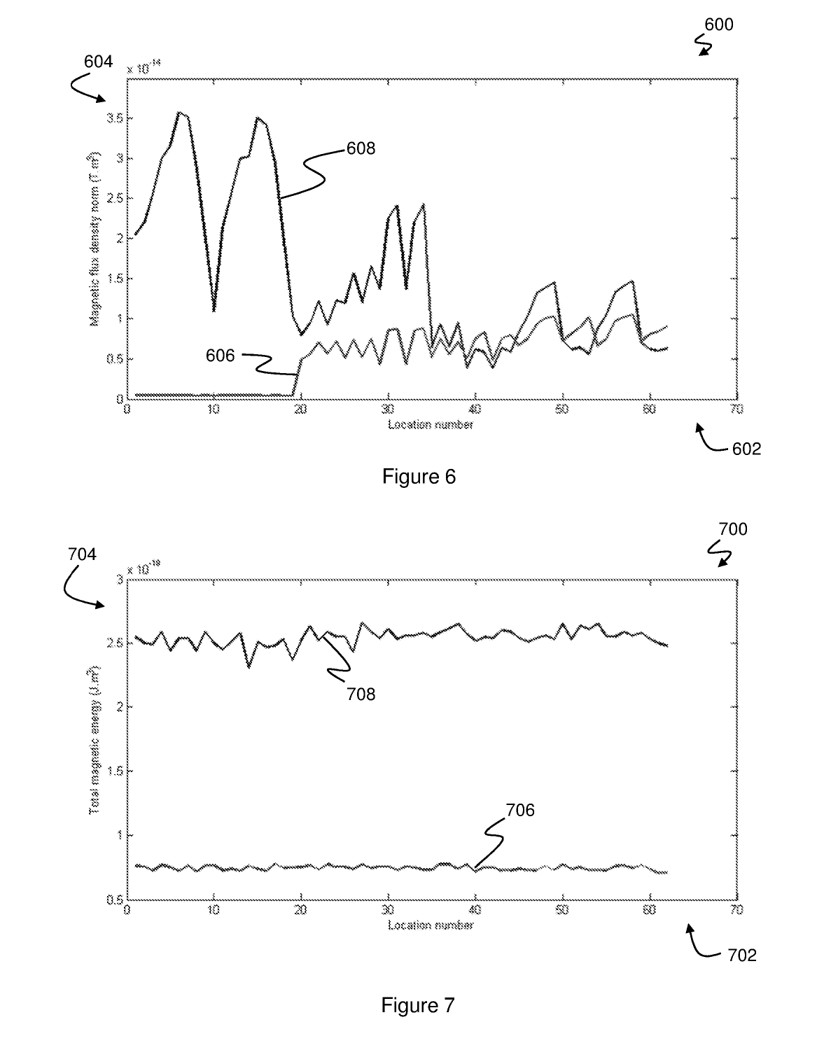

The volume within which the magnetic field exceeds the threshold value may be compared with the implanted microcoil system disclosed by Bonmassar (cited above). A number of such comparisons are illustrated in the graphs shown in FIGS. 6 to 9.

FIG. 6 is a graph 600 illustrating magnetic flux density at various points adjacent to the magnetic circuit 400, as compared with a comparable microcoil implant. The horizontal axis 602 represents a number of selected points which, for each device, are distributed within a volume adjacent to the circuit within which a concentration of magnetic field is desired. The vertical axis 604 represents magnetic flux density at each selected location. In both cases, the same electrical input power is employed. Computed results for the microcoils are shown by the line 606, while computed results for the magnetic circuit are shown by the line 608. As can clearly be seen, a much higher magnetic flux density is achieved by the magnetic circuit embodying the invention over a majority of locations within the volumes of interest.

FIG. 7 is a graph 700 illustrating total magnetic energy within various small volumes corresponding with the locations described above with reference to FIG. 6. Each of the small volumes is a sphere, within which the magnetic energy is integrated. The size of the spheres used for the microcoil and for the magnetic circuit is uniform. The horizontal axis 702 again represents each one of the different locations, while the vertical axis 704 is now total magnetic energy within the corresponding small spherical volume. The lower line 706 represents magnetic energy within the volume surrounding the microcoils, while the upper line 708 represents the magnetic energy within the volume surrounding the magnetic circuit. Over the small spherical volumes used, it can be seen that in both cases the total magnetic energy is consistent across the selected locations, and that for the same electrical input energy the magnetic circuit embodying the invention delivers over three times the total magnetic energy to the volume of interest.

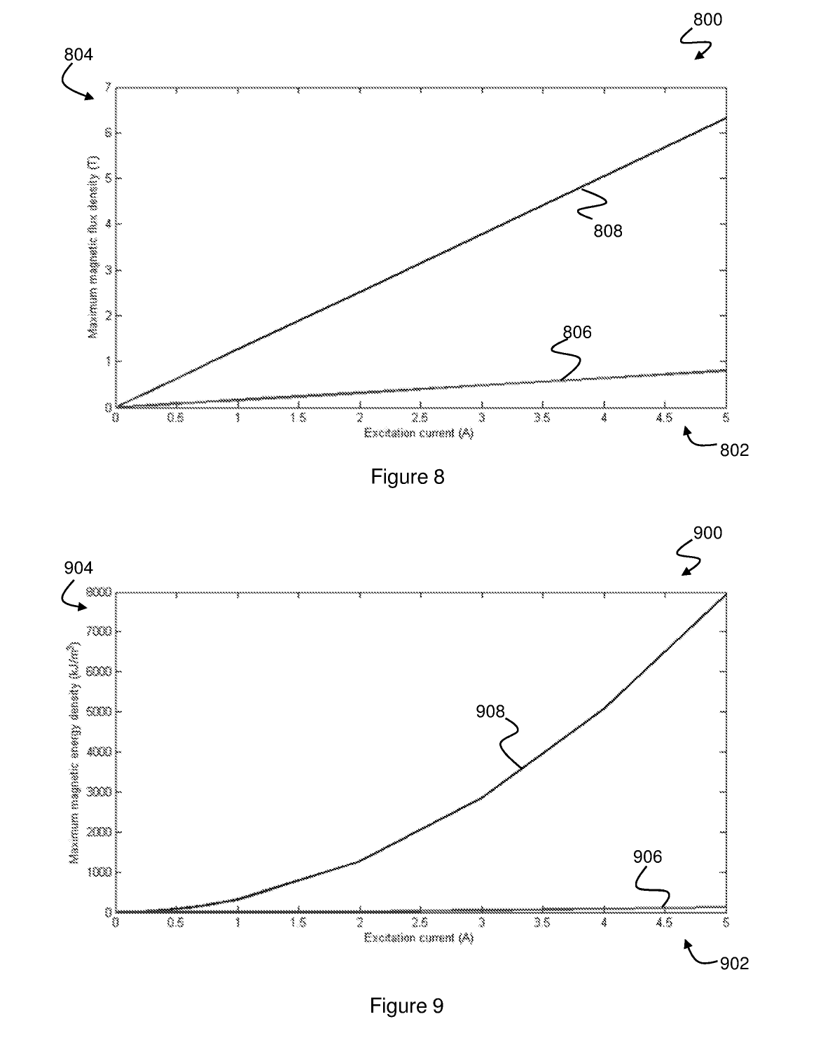

FIG. 8 is a graph 800 illustrating maximum magnetic flux density adjacent to the magnetic circuit embodying the invention, as compared with a comparable microcoil implant, as a function of excitation current. The horizontal axis 802 represents current, varying from 0 to 5 ampere, while the vertical axis 804 shows maximum magnetic flux density within the adjacent volume of interest. As can be seen, the maximum magnetic flux density available at a given excitation current for the microcoil arrangement (line 806 in the graph 800) is less than one-sixth of that of the magnetic circuit embodying the invention (line 808 of the graph 800). The ability to use significantly lower excitation current/energy with embodiments of the invention provides advantages of reduced power consumption and lower heating.

FIG. 9 is a graph 900 illustrating maximum magnetic energy density in a volume of interest adjacent to the magnetic circuit embodying the invention, as compared with a comparable microcoil arrangement, as a function of excitation current. The horizontal axis 902 shows excitation current, while the vertical axis 904 shows maximum magnetic energy density. The lower curve 906 shows the calculated maximum magnetic energy density for the microcoil arrangement, while the upper curve 910 shows the corresponding calculated values for the magnetic circuit embodying the invention. Again, a clear advantage in delivering high-density magnetic fields to a volume of interest is achieved through the use of embodiments of the invention.

The results in FIGS. 6 to 9 clearly demonstrate that, even with a reduction in efficiency of around 30% through the use of a non-superconducting diamagnetic material such as pyrolytic carbon, significant improvements over a microcoil implant may be realised.

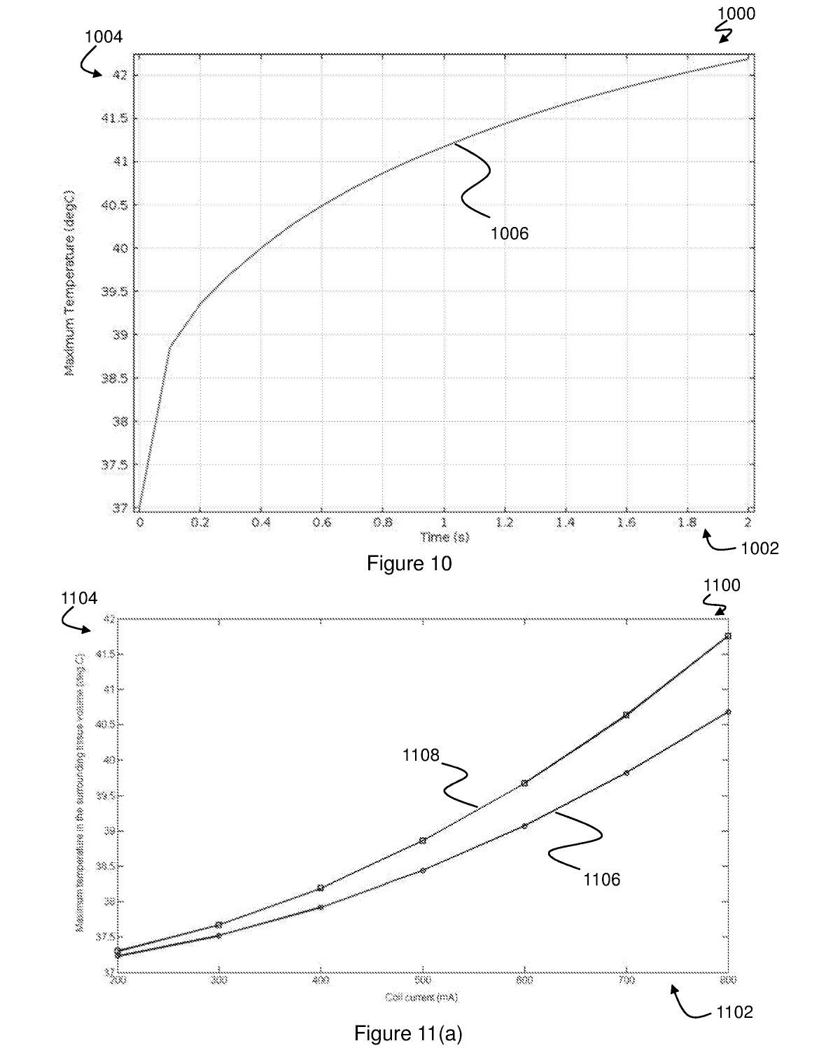

FIG. 10 is a graph 1000 illustrating maximum temperature as a function of time for which a continuous excitation current is applied to an embodiment of the invention corresponding with the structure 400 shown in FIG. 4. The current excitation is applied at 5 kHz, and 800 mA, using conductive coils having 17 turns with a cross-section of 0.00375 mm². The horizontal axis 1002 shows the time for which the excitation current is applied, between 0 and 2 seconds, while the vertical axis 1004 shows maximum temperature reached adjacent to the circuit during the operating period. For the case of an implanted device, the ambient temperature is body temperature, and a rise in temperature occurs immediately adjacent to the coils for as long as current is flowing. As can be seen, even with a full two seconds of continuous operation at 5 kHz (i.e. a total of 10,000 pulses) localised heating remains within safe levels.

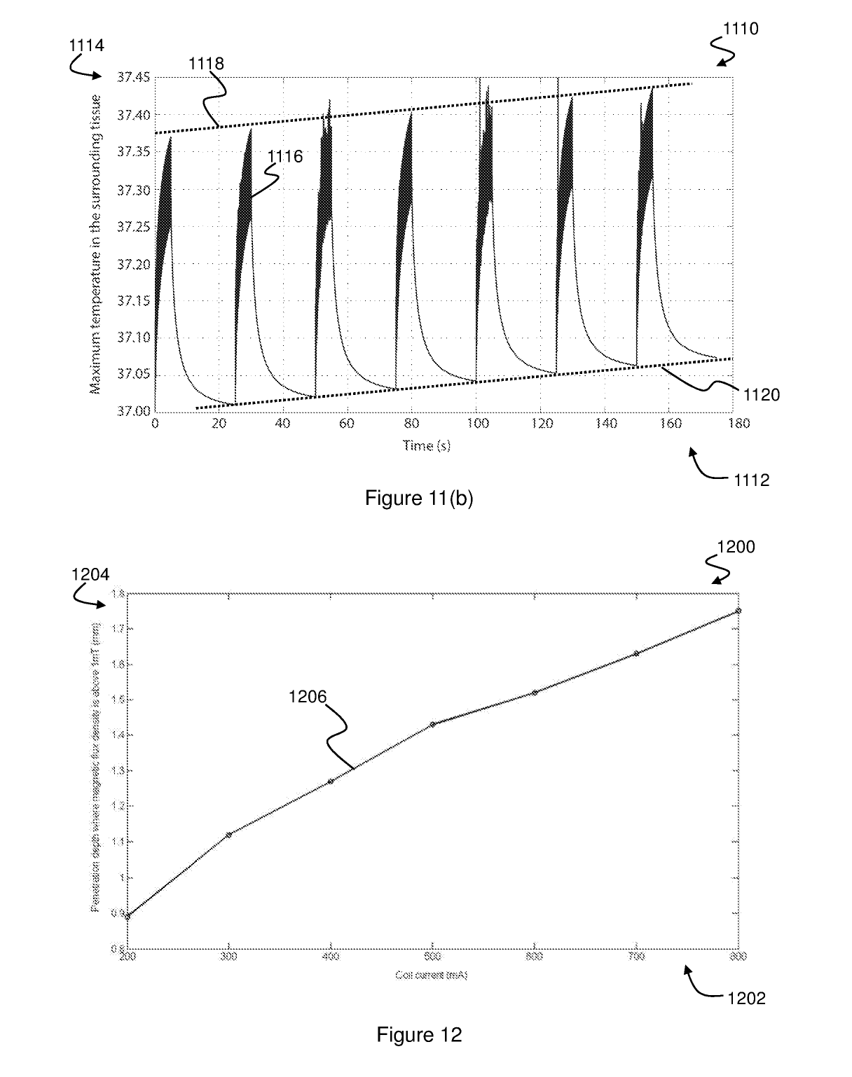

FIG. 11(a) shows a graph 1100 illustrating maximum temperature as a function of applied current, under the same conditions as for the results shown in FIG. 10, for fixed durations of one second (curve 1106) and two seconds (curve 1108). The horizontal axis 1102 shows the coil current, between 200 mA and 800 mA, while the vertical axis 1104 shows the maximum temperature reached in surrounding tissue. Again, over the range of conditions computed temperatures can be seen to remain within safe levels.

Further reductions in heating are available by using pulse train excitation with on and off periods, allowing time for cooling during the off periods. This is illustrated by the results shown in FIG. 11(b), which is a graph 1110 of temperature on the vertical axis 1114 against time on the horizontal axis 1112. Current pulses of 2 A (compared with only 800 mA in the continuous current case 1100) are applied to each coil, as 10 Hz pulse trains, e.g. 1116, over a 5 second duration. The pulse trains are separated by a 25 second ‘off’ period, during which cooling of the surrounding brain tissue occurs. The accumulation of maximum and minimum temperature over a series of seven pulse trains is linear, as shown by the lines 1118, 1120 respectively. In particular, the accumulation of temperature is 0.01° C. per pulse train. Accordingly, at the end of 20 pulse trains, corresponding with 1000 pulses as in a typical repetitive Transcranial Magnetic Stimulation (rTMS) course, the accumulated temperature rise would be only 0.2° C., and the maximum temperature within the tissue during the whole course will be 37.57° C. The heating is thus considerably lower in such an rTMS than with continuous exposure. This enables the use of higher currents to achieve stronger induced fields and increased penetration.

Furthermore, spatial calculations of temperature variation conducted by the inventors have shown that temperature rise occurs primarily very close to the coils, and decreases rapidly. Accordingly, further reductions in maximum temperature could be achieved by incorporating heat sinks around the coils to further distribute the generated heat.

FIG. 12 is a graph 1200 illustrating magnetic field penetration depth, measured at the depth at which the magnetic flux density falls below 1 mT, as a function of the current excitation level. The horizontal axis 1202 shows the applied coil current, between 200 mA and 800 mA, while the vertical axis 1204 shows the penetration depth in millimetres. As shown by the computed results 1206, it is possible to achieve penetration depths approaching 2 mm with coil currents of under 1 A.

While particular embodiments have been described in detail, it will be appreciated that these embodiments are provided by way of example only, such that the skilled person can understand the operation of the invention. Numerous variations are possible, including designs employing a single magnetic source, the provision of additional magnetic sources, and variations in the shape of the elements making up embodiments of the invention.

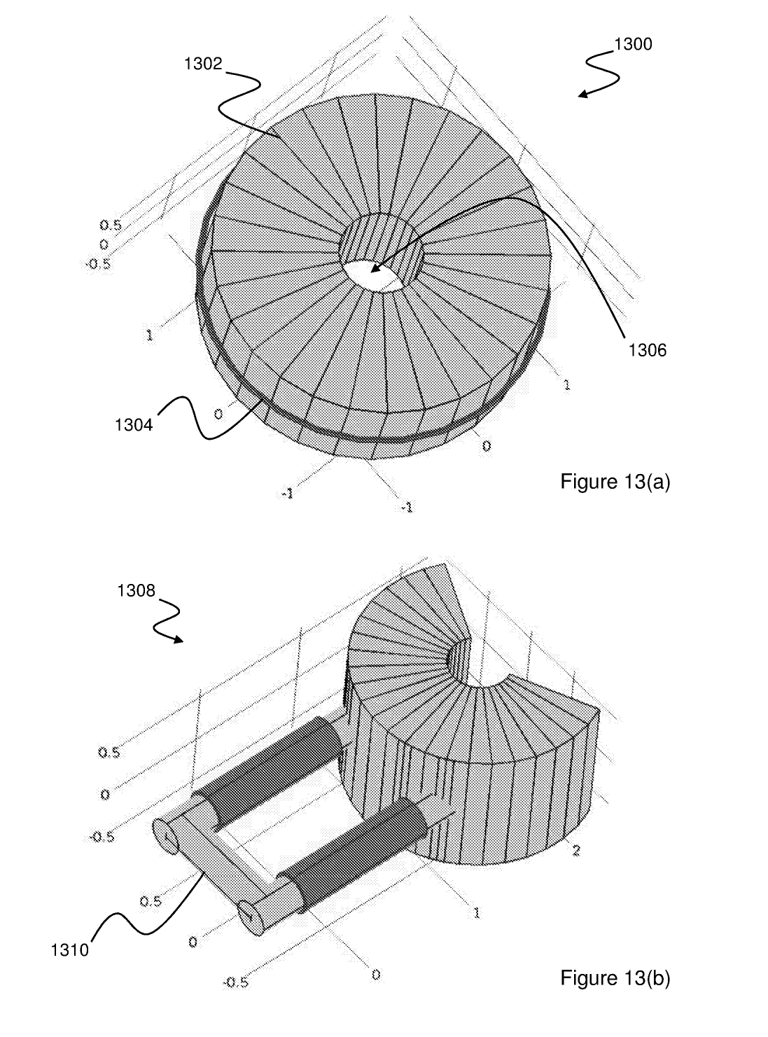

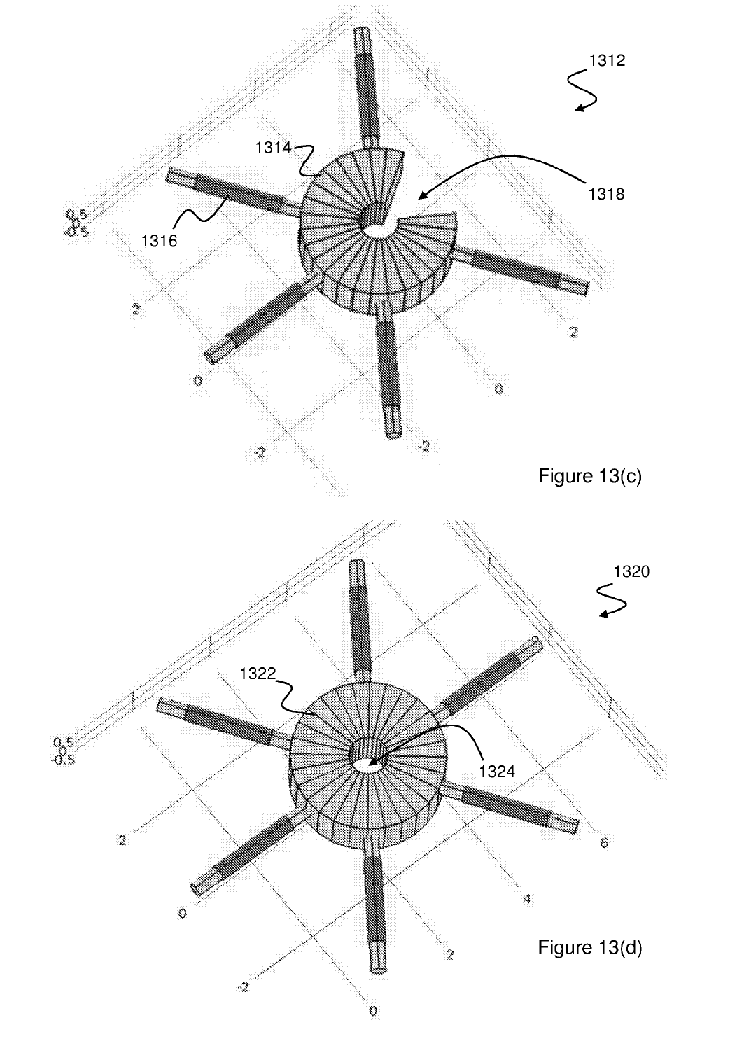

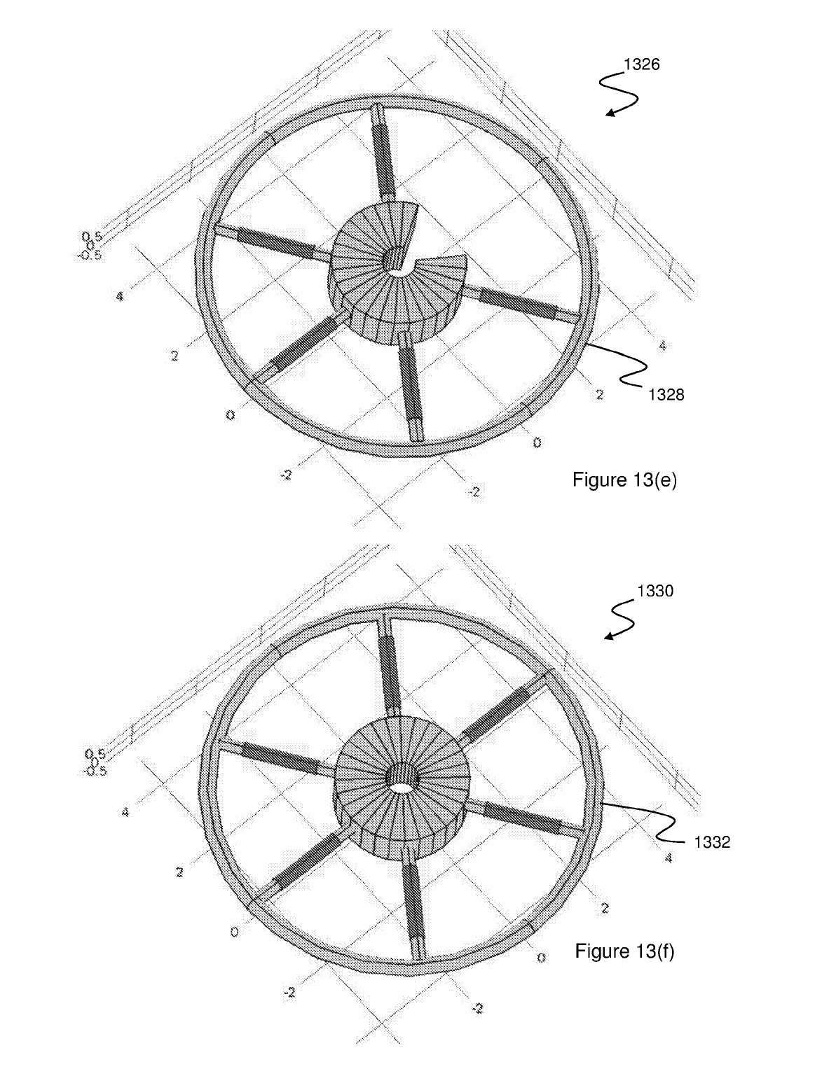

A number of such alternatives are illustrated in FIG. 13. One alternative embodiment 1300, shown in FIG. 13(a), comprises an annular flux concentrating element 1302 having a magnetic source in the form of a coil 1304 disposed around the outer circumference. In such arrangements, magnetic flux is concentrated within the central region 1306 of the annulus.

In some embodiments, for example, the ‘U’-shaped portion 110 used to couple the two magnetic sources 102, 104 may take on alternative shapes or forms. In one alternative design 1308, illustrated in FIG. 13(b), a straight connecting portion 1310 is employed. Calculations carried out by the inventors have suggested that, of the arrangements trialled, a ‘U’-shaped connecting portion 110 results in better overall performance than a straight connecting portion 1310. Nonetheless, this and other similar variations employing the principles taught by the exemplary embodiments fall within the scope of the invention.

A further embodiment 1312, shown in FIG. 13(c), comprises a C-shaped flux concentrating element 1314, to which are coupled a plurality of external, radially-oriented, magnetic sources, e.g. 1316, whereby flux is concentrated within the gap 1318 of the C-shaped element. In yet another related arrangement 1320, the C-shaped element is replaced with an annular flux concentrating element 1322, such that flux is concentrated within the central region 1324 of the annulus.

Yet another embodiment 1326 is illustrated in FIG. 13(e). The embodiment 1326 is similar to the embodiment 1312, except that the radial sources 1316 are magnetically-coupled at their outer ends by an annular conducting portion 1328. In like manner, the embodiment 1330 shown in FIG. 13(f) is similar to the embodiment 1320 with the inclusion of annular conducting portion 1332.

Overall, embodiments of the invention offer a number of potential advantages, in appropriate applications, over prior art means for generating localised magnetic fields. For example, embodiments of the invention are able to concentrate a magnetic field within a small volume, enabling enhanced electric fields to be induced within the target volumes, while reducing effects outside the target volumes. The features embodying the invention and its variations which achieve these benefits are not limited to those disclosed above and depicted in the accompanying drawings, but rather are as defined by the scope of the claims appended hereto.

The claims defining the invention are as follows:

1. A magnetic circuit comprising a magnetic path including:
at least one magnetic source arranged to generate magnetic flux within the magnetic path; and
at least one magnetic flux-concentrating element, magnetically coupled to the magnetic source, and arranged to concentrate magnetic-flux generated by the magnetic source within a volume adjacent to the magnetic flux-concentrating element.

2. The magnetic circuit of claim 1 wherein the at least one magnetic source comprises at least first and second magnetic sources.

3. The magnetic circuit of claim 2 wherein the first and second magnetic sources are arranged to generate magnetic flux in a common direction within the magnetic path.

4. The magnetic circuit of claim 2 wherein the first and second magnetic sources are magnetically coupled by a continuous length of magnetic material defining a portion of the magnetic path.

5. The magnetic circuit of claim 4 wherein the continuous length of magnetic material defines a portion of the magnetic path comprising a substantially 180 degree change of direction of magnetic flux.

6. The magnetic circuit of claim 5 wherein the continuous length of magnetic material comprises a substantially ‘U’-shaped section.

7. The magnetic circuit of claim 2 wherein the magnetic flux-concentrating element comprises first and second tapered portions, each formed from a magnetic material and having a wider end and a narrower end, wherein the wider end of the first tapered portion is magnetically coupled to the first magnetic source, the wider end of the second tapered portion is magnetically coupled to the second magnetic source, and the narrower ends of the first and second tapered portion converge within a volume in which magnetic flux is concentrated.

8. The magnetic circuit of claim 6 wherein the concentrating element further includes at least one diamagnetic portion located adjacent to the first and second tapered portions.

9. The magnetic circuit of claim 2 in which the magnetic flux-concentrating element comprises a structure forming an arc having an inner surface and an outer surface wherein:
the first and second magnetic sources are magnetically coupled to the outer surface of the structure; and
the structure comprises an alternating arrangement of magnetic and diamagnetic materials forming layers extending between the outer surface and the inner surface.

10. The magnetic circuit of claim 9 wherein the arc is a substantially circular arc, such that the inner surface is defined by an inner radius, and the outer surface is defined by an outer radius.

11. The magnetic circuit of claim 10 wherein the arc is an open arc, such that the structure is substantially ‘C’-shaped.

12. The magnetic circuit of claim 9 wherein the diamagnetic material comprises pyrolytic carbon.

13. A method of generating a magnetic field which comprises:
providing a magnetic circuit according to any one of the preceding claims; and
applying driving electrical signals to the first and second magnetic sources in order to generate a magnetic flux within the magnetic path, whereby a concentration of magnetic flux is generated within a volume adjacent to the magnetic flux-concentrating element.
